(19) United States
(12) Patent Application Publication
(10) Pub. No.: US 2013/0224226 A1
(43) Pub. Date: Aug. 29, 2013

(54) INSECT BINDING ANTIBODIES

(71) Applicants: Peter Verheesen, Mariakerke (BE); Erik Jongedijk, Lokeren (BE)

(72) Inventors: Peter Verheesen, Mariakerke (BE); Erik Jongedijk, Lokeren (BE)

(73) Assignee: AGROSAVFE N.V., Gent (BE)

(21) Appl. No.: 13/819,326

(22) PCT Filed: Aug. 25, 2011
(86) PCT No.: PCT/EP2011/064663

§ 371 Date: May 11, 2013

Related U.S. Application Data
(60) Provisional application No. 61/402,303, filed on Aug. 26, 2010.

(30) Foreign Application Priority Data
Sep. 7, 2010 (EP) 10175592.4

(51) Int. Cl. C07K 16/18; A01N 37/44

(52) U.S. Cl. CPC C07K 16/18; A01N 37/44
USPC 424/178.1; 530/389.1; 530/391.7

(57) ABSTRACT

Described is a binding domain, preferably an antigen binding domain, more preferably an antigen binding domain that specifically binds a binding site on an insect. More specifically, described are antigen binding domains comprising an amino acid sequence that comprises 4 framework regions and 3 complementary determining regions, whereby the antigen binding domains are capable to bind specifically to an insect as a whole, preferably to a binding site on the insect surface. Further described is the use of the binding domain to deliver a compound, preferably a biologically active agent to the insect, and to insecticidal compositions comprising the binding domain.

### CROSS-REFERENCE TO RELATED APPLICATIONS

This application is a national phase entry under 35 U.S.C. §371 of International Patent Application PCT/EP2011/064663, filed Aug. 25, 2011, designating the United States of America and published in English as International Patent Publication WO 2012/025602 A1 on Mar. 1, 2012, which claims the benefit under Article 8 of the Patent Cooperation Treaty and under 35 U.S.C. §119(e) to U.S. Provisional Patent Application Ser. No. 61/402,303, filed Aug. 26, 2010, and to European Patent Application Serial No. 10175592.4, filed Sep. 7, 2010.

### TECHNICAL FIELD

The disclosure relates to a binding domain, preferably an antigen binding domain, more preferably an antigen binding domain that specifically binds a binding site on an insect. More specifically, it relates to antigen binding domains comprising an amino acid sequence that comprises 4 framework regions and 3 complementary determining regions, whereby the antigen binding domains are capable to bind specifically to an insect as a whole, preferably to a binding site on the insect surface. Further described is the use of the binding domain to deliver a compound, preferably a biologically active agent to the insect, and to insecticidal compositions comprising the binding domain.

### BACKGROUND

Insecticides can be classified in systemic insecticides and contact insecticides. Systemic insecticides exert their action when the insect is feeding on the host; for contact insecticides, insects are killed by simple contact of the insect with the insecticide. In any case, for pesticide application and especially for contact insecticide application, it is extremely important to ensure that the contact between the insect and the insecticide is possible, while limiting the amount of insecticide sprayed and minimizing the contamination of the environment. To realize this goal, controlled droplet application received extensive research interest, although the results were not always satisfying. Depending on the method of application and climatic factors as much as 90% of conventionally applied agrochemicals never reach their objectives: to produce the desirable biological response at the precise time and in the quantities required (Kenawy, 1998).

To obtain improved product performance, controlled release technologies have been developed. Controlled release is a method whereby active ingredients are made available to a specified target at a certain concentration and duration to produce an intended effect. In a controlled release formulation, the initial levels of the pesticide are chosen in order to maintain the pesticidal concentration above the minimum inhibitory concentration for the pest, until the end of the desired period of effectiveness, without unneeded dispersal in the environment. Contained release is normally realized by coating granules with pesticides, by binding pesticides on a polymeric carrier, by entrapping pesticides in a polymeric matrix or by micro-encapsulation. Especially the micro-encapsulation technology enables the manufacturer to develop a formulation with reduced toxicity and workers exposure, with timed and controlled activity, with reduced evaporation losses, reduced phytotoxicity, controlled environmental degradation, reduced leaching into the groundwater and reduced levels in the environment. Microencapsulated insecticides have been shown to ‘stick’ to a certain extent to the insect, most probably due to aspecific interactions between the microcapsule shell and the insect body. This sticking improves the contact between insecticide and insect and as such may contribute to the efficacy of the microencapsulated active ingredient (Perrin, 2000).

Another approach to bring insects more efficiently in contact with insecticides is the so-called “attract-and-kill” strategy. In these cases, the insecticide is combined with an attracting agent such as a pheromone; to attract the insect to the insecticide. However, although this is certainly an interesting approach, the results have been disappointing in some cases due to insufficient contact of the insects with the contact insecticide (Knight, 2010).

The contact between the insect and the insecticide could be dramatically improved by coupling the insecticide, or a carrier, whereon or wherein the insecticide is contained, to a molecule binding specifically to the insect, such as an insect binding domain. Such an insect binding domain is preferably a domain that binds to the insect as a whole or to a part of the insect body which is accessible from the outside, preferably part of the insect surface or its exoskeleton.

An important structural component of the insect exoskeleton is chitin. Chitin binding domains are known to the person skilled in the art and can be derived from chitinases (Iseli et al., 1993; Hamel et al., 1997) or from cuticular proteins from arthropods (Rebers and Willis, 2001). The industrial use of chitin binding domains is rather limited: US200619925 discloses a mutant chitin binding domain and its use for protein purification; US5187262 discloses the use of a chitin binding domain in fungal growth inhibition. However, there are no data that indicate that those domains are capable of binding the surface of intact living insects and in doing so, delivering compounds, preferably biologically active agents to the insect. Chitin antibodies have been disclosed in US5004699 and WO2009069007; those antibodies can be used for the detection of fungi and yeasts (US5004699), or, in combination with anti-laminarin antibodies, for the diagnosis and prognosis of Crohn's disease (WO2009069007); Sales et al. (2001) disclose an anti-chitin antibody, useful for immunolabeling of insect midgut microtome sections. Again, in none of those cases, evidence is presented that such antibodies would be capable of binding (the surface of) intact insects and in doing so, delivering compounds, preferably biologically active agents to the insect. This may be explained by the fact that chitin is predominantly present in the inner procuticle layer of the exoskeleton, while the outer epicuticular layer contains little or no chitin.

Doctor et al. (1985) disclose antibodies, binding pupal cuticle proteins. Although, in principle, the pupal cuticle proteins could be an interesting target for in vivo targeting, antibody binding was only demonstrated on 4% formalin treated preparations, and no binding on intact insects was disclosed. As the antibodies have been generated using urea extraction, one can assume that the epitopes are not accessible in a native confirmation of the protein and that therefore these antibodies could not be used to bind and retain a compound to an insect.

There is still need for a binding domain, capable of binding a binding site situated on the outside surface of insects, preferably of intact insects, more preferably of living intact insects, most preferably the binding domain is capable of binding a binding site on the exoskeleton of living intact insects and, in doing so, can be used for delivering and/or retaining a compound, preferably a biologically active agent to the insect.

### SUMMARY OF THE DISCLOSURE

Surprisingly, we succeeded in isolating antigen binding domains from llamas, immunized with complex insect homogenates by selecting antigen binding domains on entire, intact insects. The antigen binding domains are capable of binding to the insect surface and can be used to bind and retain a compound, preferably a biologically active agent, to the insect.

### BRIEF DESCRIPTION OF THE FIGURES

FIG. 1: Schematic outline of a whole insect ELISA.

FIG. 2: Binding of anti-insect VHH to adult aphids.

Pea aphids were labeled with VHH and bound VHH were detected with mouse anti-histidine antibodies and rabbit anti-mouse IgG conjugated with Alexa594. Clear binding of VHH to pea aphid abdomen was observed.

FIG. 3: Targeting of microcapsules with coupled anti-insect VHH to insects.

Whole pea aphids were incubated with microcapsule solutions and non-bound microcapsules were removed by washes and removing supernatants. Bound microcapsules were visualized using epifluorescence microscopy. Pictures are showing a combined image of bright field microscopy with detection of bound microcapsules by epifluorescence. Microcapsules appear as black dots in this figure.

### DETAILED DESCRIPTION

Binding Domains

A first aspect hereof is a binding domain, more preferably an antigen binding domain, capable of binding a binding site on an insect, preferably on an intact insect, even more preferably on an intact living insect. Most preferably, the binding site is situated on the insect surface, on a part of the insect body that is accessible from the outside, such as, but not limited to the exoskeleton of the intact living insect. A “binding site,” as used herein, means a molecular structure or compound, such as a protein, a peptide, a polysaccharide, a glycoprotein, a lipoprotein, a fatty acid, a lipid or a nucleic acid or a particular conformation of such molecular structure or compound, or a combination or complex of such molecular structures or compounds; preferably the binding site is comprised in an insect structure, such as but not limited to head, thorax, abdomen, trachea, spiracles, antennae, insect legs and/or claws, wings, wingshells, mouthparts and eyes. Even more preferably, the binding site comprises at least one antigen. An “antigen,” as used herein, is a molecule capable of eliciting an immune response in an animal.

Binding of the binding domain to the binding site is a specific binding; aspecific sticking of a compound to the insect is not considered as binding of a binding domain to a binding site. In principle, the binding site can be situated anywhere on the insect, as long as the binding domain can bind and thereby is capable of delivering and/or retaining a compound, preferably a biologically active agent, to the insect.

“Binding domains” are known to the person skilled in the art and include, but are not limited to carbohydrate binding domains and antigen binding domains, such as those in heavy chain antibodies (hcAb), single domain antibodies (sdAb), minibodies (Tramontano et al., 1994), the variable domain of camelid heavy chain antibodies (VHH), the variable domain of the new antigen receptors (VNAR; Nutall et al., 2003), engineered CH2 domains (nanoantibodies; Dimitrov, 2009) and alphabodies (WO2010066740). Preferably, the binding domain is an antigen binding domain. An “antigen binding domain,” as used herein, is a binding domain that binds to an antigen. Preferably, the antigen binding domain is comprised in an antigen binding domain comprising an amino acid sequence that comprises 4 framework regions and 3 complementary determining regions, according to Kabat. Binding domains comprising 4 FRs and 3 CDRs, preferably in a sequence FR1-CDR1-FR2-CDR2-FR3-CDR3-FR4, are known to the person skilled in the art and have been described, as a non-limiting example in Wesolowski et al. (2009). The length of the CDR3 loop is strongly variable and can vary from 0, preferably from 1, to more than 20 amino acid residues, preferably up to 25 amino acid residues. Preferably, the antigen binding domains are derived from camelid antibodies, preferably from heavy chain camelid antibodies, devoid of light chains, such as variable domains of heavy chain camelid antibodies (VHH). Camelid antibodies, and the VHH derived sequences are known to the person skilled in the art. Camelid antibodies have been described, amongst others in WO9404678 and in WO2007118670. Those antibodies are easy to produce, and are far more stable than classical antibodies, which provides a clear advantage for stable binding to the insect in a natural environment, where the binding conditions cannot be controlled.

Most preferably, the VHH comprises, preferably consists of a sequence selected from the group consisting of SEQ ID NO:1-SEQ ID NO:5, or any suitable fragment thereof (which will then usually contain at least some of the amino acid residues that form at least one of the complementary determining regions) or homologues thereof. Homologues, as used herein, are sequences wherein each or any framework region and each or any complementary determining region shows at least 80% identity, preferably at least 85% identity, more preferably 90% identity, even more preferably 95% identity with the corresponding region in the reference sequence (i.e., FR1_homologue versus FR1_reference, CDR1_homologue versus CDR1_reference, FR2_homologue versus FR2_reference, CDR2_homologue versus CDR2_reference, FR3_homologue versus FR3_reference, CDR3_homologue versus CDR3_reference and FR4_homologue versus FR4_reference) as measured in a BLASTp alignment (Altschul et al., 1997; FR and CDR definitions according to Kabat).

Insects

An “insect,” as used here, is used in the broad popular sense and includes all species of the superphylum Panarthropoda (classification Systema Naturae, Brands, S. J. (comp.) 1989-2005. Systema Naturae 2000. Amsterdam, The Netherlands. [http://sn2000.taxonomy.nl/]), including the phyla Arthropoda, Tardigrada and Onychophora; it includes all the different phases of the life cycle, such as, but not limited to eggs, larvae, nymphs, pupae and adults. Preferably, the insect belongs to the phylum Arthropoda (including, but not limited to the orders Archaeognatha, Thysanura, Paleoptera and Neoptera, also ticks, mites and spiders), even more preferably to the epiclass Hexapoda, most preferably to the class Insecta. Preferably, the insect is considered as a pest. A “pest,” as used here, is an organism that is detrimental to humans or human concerns, and includes, but is not limited to agricultural pest organisms, including but not limited to aphids, grasshoppers, caterpillars, beetles, etc., household pest organisms, such as cockroaches, ants, etc., and disease vectors, such as malaria mosquitoes. More preferably, the insect is an agricultural pest organism, even more preferably, the insect is an aphid. An “intact living insect” refers to the insect as it occurs in its natural habitat.

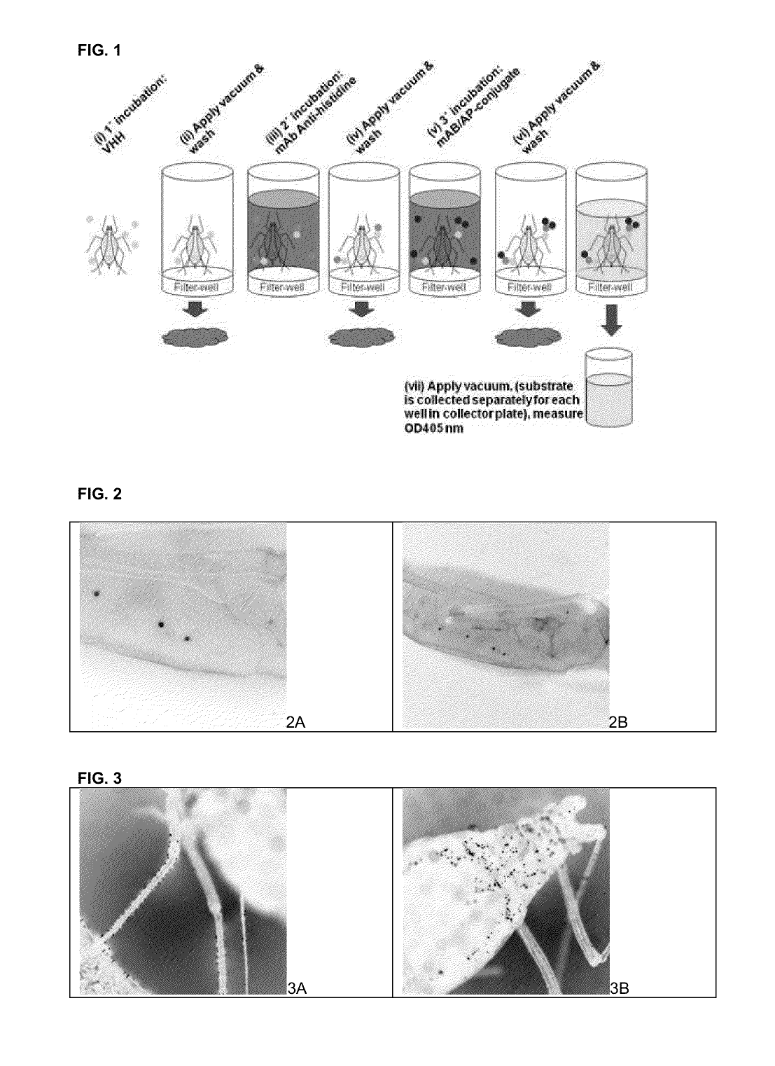

“Capable of binding a binding site on an insect,” as used here, means that the binding domain can bind to a binding site on an insect, preferably on an intact living insect, more preferably on the insect surface, most preferably on the insect exoskeleton, without special preparation of the insect tissue. Insect binding can be tested by a whole insect ELISA assay, as shown in FIG. 1, and as exemplified in more detail in Example 3.

An “insect surface,” as used herein, can be any surface as it occurs on the outside of an insect as defined above; however, it excludes histological preparations of insects. Preferably, the insect surface is the surface of an insect structure selected from the group consisting of trachea, spiracles, antennae, insect legs and/or claws, wings, wingshells, mouthparts and eyes. Preferably, the binding domain is capable of binding to the insect surface under conditions that are reminiscent of conditions in the field or in a greenhouse or in a human inhabited environment. More preferably, the binding domain is maintaining its binding functionality in a pesticide formulation and/or in an agrochemical formulation (both as defined hereinafter).

Another aspect hereof is the binding domain capable of retaining a carrier, preferably a microcarrier, on an insect. “Retaining,” as used herein, means that the binding force resulting from the affinity or avidity of either one single binding domain or a combination of two or more binding domains for its or their antigen is larger than the combined force and torque imposed by the gravity of the carrier and the force and torque, if any, imposed by shear forces caused by one or more external factors; “retaining” can be evaluated by the fact that the contact between the carrier and the insect is better (expressed in time of contact, number of carriers per insect, or distance between the carrier and the insect) for a carrier with binding domain, compared with a carrier without binding domain, when applied under identical conditions. “Capable of retaining a carrier on an insect,” as used here, means that the binding force of the binding domain is strong enough to retain a carrier to a binding site on an insect, preferably on an intact living insect, most preferably on the insect surface (as defined above) of an intact living insect. Preferably, the insect surface is the surface of an insect structure selected from the group consisting of trachea, spiracles, antennae, insect legs and/or claws, wings, wingshells, mouthparts and eyes. Preferably, the binding domain is capable of binding to the insect surface under conditions that are reminiscent of conditions in the field or in a greenhouse or in a human inhabited environment. More preferably, the binding domain is maintaining its binding functionality in a pesticide formulation and/or in an agrochemical formulation (both as defined hereinafter). Preferably, the binding domain, more preferably the antigen binding domain, is comprised in an amino acid sequence that comprises 4 framework regions and 3 complementary determining regions, according to Kabat. Even more preferably, the antigen binding domains are derived from camelid antibodies, preferably from heavy chain camelid antibodies, devoid of light chains, such as, variable domains of heavy chain camelid antibodies (VHH). Most preferably, the VHH comprises, preferably consists of a sequence selected from the group consisting of SEQ ID NO:1-SEQ ID NO:5, or any suitable fragment thereof (which will then usually contain at least some of the amino acid residues that form at least one of the complementary determining regions) or homologues thereof.

“Carrier,” as used herein, means a solid or semi-solid vehicle in or on(to) which a compound, preferably a biologically active agent can be suitably incorporated, included, immobilized, adsorbed, absorbed, encapsulated, embedded, attached, etc., such as microcapsules, nanocapsules, liposomes, vesicles, polymers (e.g., in the form of (nano- or micro-) spheres, beads, a gel, small particles, small granules, a sheet or any other suitable form) such as, for example, urethane, poly-urea, poly-ethylene, polyethylene-glycol, polyvinyl alcohols, melamine formaldehyde, acrylic polymers, vinyl acetate or siloxane polymers or, optionally, (and usually preferably) for agrochemical purposes, biodegradable polymers (such as, for example, agar, gelatin, alginates, pectins, poly-alcohols such as cetyl-alcohol, oily substances such as hydrogenated palm oil or soybean oil, starches, waxes etc.), water droplets that are part of an water-in-oil emulsion, oil droplets that are part of an oil-in-water emulsion, inorganic materials such as talc, clay, microcrystalline cellulose, silica, alumina, silicates and zeolites, a gel, or even microbial cells (such as yeast cells) or suitable fractions or fragments thereof (as further described herein). It is also possible, that one or more compounds are either present on or within a microbial cell or a phage (for example, because the one or more compounds can be loaded into (or onto) such cells or are biologicals that have been produced/expressed in the microbial cell) or that the one or more compounds are associated (e.g., bound to or embedded in) with cell fragments (e.g., fragments of cells walls or cell membranes), cell fractions or other cell debris (for example, obtained by fractionating or lysing the microbial cells into (or onto) which the one or more compounds have been loaded, produced or expressed). In the case of a microbial cell or phage, the targeting agent comprising at least one antigen binding protein according to the invention may be encoded by the microbial cell or phage genome, whereas the compound is contained in or coupled to the phage, either as fusion protein or by chemical linking. Other suitable carriers will be clear to the skilled person based on the disclosure herein. Preferably, the carrier is such that the one or more biologically active agent can be incorporated, encapsulated or included into the carrier, e.g., as a nanocapsule, microcapsule, nanosphere, micro-sphere, liposome or vesicle. Preferably the carriers are such that they have immediate or gradual or slow release characteristics, for example, over several minutes, several hours, several days or several weeks. Also, the carriers may be made of materials (e.g., polymers) that rupture or slowly degrade (for example, due to prolonged exposure to high or low temperature, sunlight, high or low humidity or other environmental factors or conditions) over time (e.g., over minutes, hours, days or weeks) and so release the biologically active agent from the carrier.

A “microcarrier,” as used herein, is a particulate carrier where the particles are less than 500 μm in diameter, preferably less than 250 μm, even more preferable less than 100 μm, most preferably less than 50 μm. Such microcarriers have been described, amongst others, in US6180141, WO2004004453, WO2005102045 and US7494526.

Preferably, the carrier is coupled, bound, linked or otherwise attached to or associated with one or more binding domains according to the invention. More preferably, the carrier is covalently coupled to the binding domain.

A further aspect hereof is a binding domain according to the invention, which is capable of retaining a compound on an insect. “Capable of retaining a compound on an insect,” as used here, means that the binding force of the binding domain is strong enough to retain a compound to a binding site on an insect, preferably on a intact living insect, most preferably on the insect surface (as defined above) of an intact living insect. Preferably, the insect surface is the surface of an insect structure selected from the group consisting of trachea, spiracles, antennae, insect legs and/or claws, wings, wingshells, mouthparts and eyes. Preferably, the binding domain is capable of binding to the insect surface under conditions that are reminiscent of conditions in the field or in a greenhouse or in a human inhabited environment. More preferably, the binding domain is maintaining its binding functionality in a pesticide formulation and/or in an agrochemical formulation (both as defined hereinafter). Preferably, the binding domain, more preferably the antigen binding domain, is comprised in an amino acid sequence that comprises 4 framework regions and 3 complementary determining regions, according to Kabat. Even more preferably, the antigen binding domains are derived from camelid antibodies, preferably from heavy chain camelid antibodies, devoid of light chains, such as variable domains of heavy chain camelid antibodies (VHH). Most preferably, the VHH comprises, preferably consists of a sequence selected from the group consisting of SEQ ID NO:1-SEQ ID NO:5, or any suitable fragment thereof (which will then usually contain at least some of the amino acid residues that form at least one of the complementary determining regions) or homologues thereof.

A “compound,” as used herein, can be any compound, including but not limited to, proteins and protein complexes such as enzymes, or chemical compounds, including but not limited to, agrochemicals, such as, but not limited to, pesticides, growth regulators, nutrients/fertilizers, repellants, defoliants, anti-fouling agents, herbicides, fungicides and insecticides, or a combination of those, both for field use and for household and greenhouse use. Preferably, the compound is a biologically active agent. A “biologically active agent” is a compound influencing the life cycle, function and/or behavior of one or more living organisms. Biologically active agent includes, but is not limited to, toxins, hormones, growth regulators, attractants and repellents. Preferably, the biologically active agent is an insecticide. An “insecticide,” as used herein, refers to compounds having biological activity on insects (as defined above), including but not limited to compounds capable of killing the insect, larvaecides, insect growth regulators, behavior modifying compounds, attractants, repellents, pheromones, kairomones, allomones and entomopathogenic fungi, viruses and proteins. The insecticide exerts its biological activity preferably by the contact of the compound with the insect, without the need of being ingested by the insect. It includes not only compounds or compound formulations that are ready to use, but also precursors in an inactive form, which may be activated by outside factors. Possibly, the insecticide may be combined with materials used in conjunction, such as synergists or safeners, flavor or odor compositions.

Preferably, the compound is comprised in a carrier as defined above. “Comprised in a carrier,” as used herein, means bound on or contained in by means, such as, but not limited to, embedding, encapsulation and adsorption. Preferably, the compound, more preferably the carrier comprising the compound, is coupled, bound, linked or otherwise attached to or associated with one or more binding domains according to the invention. More preferably, the compound, most preferably the carrier comprising the compound, is covalently coupled to the binding domain.

Another aspect hereof is the binding domain coupled to an insecticide. The insecticide may be coupled directly to the binding domain according to the invention or, preferably, it may be bound on or comprised in a carrier. “Coupled,” as used herein, can be any coupling allowing the delivery and retention of the insecticide or carrier comprising the insecticide by the binding domain; it can be a covalent as well as affinity binding. “Affinity binding,” as used herein, includes but is not limited to, specific binding, such as, antigen-antibody interactions or lectin-polysaccharide interaction as well as non-specific interactions, such as, hydrophobic, hydrophilic, lipophilic or Van der Waals interactions. Preferably, the coupling is a covalent binding. It is clear to the person skilled in the art how binding domains can be coupled to any type of functional groups present at the outer surface of a carrier. Functional group, as used herein, means any chemical group to which a protein can be covalently bound, including but not limited to, carboxyl-, amine-, hydroxyl-, sulfhydryl-, or alkyn-group. As a non-limiting example, coupling by EDC activation of carboxylgroups can be applied. Binding domains can be coupled with or without spacers to the carrier. Examples of such spacers can be found in WO0024884 and WO0140310.

Another aspect hereof is the use of a targeting agent comprising at least one binding domain hereof to deliver, preferably to deliver and retain a compound or a combination of compounds to an insect. A “targeting agent,” as used herein, is a molecular structure, preferably a polypeptide, comprising at least one antigen binding protein. A targeting agent in its simplest form consists of one single binding domain; however, a targeting agent can comprise more than one binding domain and can be monovalent or multivalent and monospecific or multispecific, as further defined. Preferably, the compound is a biologically active agent, even more preferably the compound is an insecticide. Preferably, the insecticide is comprised in a carrier, as described above. Preferably, the insect is an intact living insect, even more preferably the insecticide is delivered to the insect surface. In a preferred embodiment, the insecticide is a contact insecticide. Retaining the insecticide on the insect has the advantage that the insect, when applicable, may take the insecticide to its nest, thereby increasing the efficacy of the insecticide in controlling the insect population. The insecticide that needs to be delivered can be comprised in a slow delivery carrier, ensuring release of the insecticide to the insect once the insect has left the place of contact.

Another aspect hereof is the use of a binding domain according to the invention to alter the target specificity of or to induce target specificity for an insecticide by coupling the insecticide, preferably comprised in a carrier, to a targeting agent, comprising at least one binding domain according to the invention that binds specifically to one particular insect species. “Target specificity,” as used herein, means the spectrum of targets on which the compound is binding under conditions of normal use. This may be particularly advantageous to reduce off-target effects on other, potentially harmless, species, of a particular insecticide.

Still another aspect hereof is a composition, comprising (i) a targeting agent comprising at least one binding domain according to the invention and (ii) a carrier as described above. Preferably, the targeting agent and carrier are coupled by affinity binding or by covalent binding. Preferably, the targeting agent is coupled to the carrier by covalent binding. Preferably, the targeting agent is coupled, preferably covalently coupled, to the carrier by the use of a functional group present on the outer surface of the carrier. Preferably, the carrier further comprises one or more insecticides, as defined earlier. The compositions hereof generally comprise at least (a) one or more insecticides, which are preferably present in or on a suitable carrier; and (b) at least one targeting agent comprising at least one binding domain according to the invention which is coupled, bound to, linked to or otherwise associated with the insecticide and/or the carrier, preferably with the carrier. Optionally, the composition further comprises one or more further components, such as, but not limited to, diluents, solvents, adjuvants, surfactants, wetting agents, spreading agents, oils, stickers, penetrants, buffering agents, acidifiers, defoaming agents, drift control agents etc., suitable for use in the composition according to the invention.

Also, as further described herein, both the covalently bound targeting agents as well as the affinity-bound targeting agents may either be a “mono-specific” targeting agent or a “multi-specific” targeting agent. By a “mono-specific” targeting agent is meant a targeting agent that comprises either a single binding domain or two or more identical binding domains, or that comprises two or more different binding domains that each are directed against the same antigen present at or in the same binding site or that form the binding site. Thus, a mono-specific targeting agent is capable of binding to a single binding site, either through a single binding domain or through multiple binding domains. By a “multi-specific” targeting agent is meant a targeting agent that comprises two or more binding domains that are each directed against different antigens present at or in a binding site or that form the binding site. Thus, a “bi-specific” targeting agent is capable of binding to two different antigens present at or in a binding site or that form the binding site; a “tri-specific” targeting agent is capable of binding to three different antigens present at or in a binding site or that form the binding site; and so on for “multi-specific” targeting agents. Accordingly, in one aspect, the above compositions hereof comprise two or more identical or different targeting agents, by which is meant two or more targeting agents that, for identical targeting agents, each bind to identical or different antigens present at or in the same binding site, whereas for different targeting agents, at least one binds to different antigens present at or in the same binding site or in different binding sites. By binding different binding sites, the effect of the insecticide coupled to the binding domain can be increased, by targeting the insecticide to different insect structures. Alternatively, the multi-specific targeting agents may be directed to, as a non-limiting example, one or more antigens present at or in a binding site on a plant at one hand, and to one or more antigens present at or in a binding site on an insect at the other hand, thereby ensuring the insecticide comprising carriers are accumulated on places often frequented by insects. Also, in respect of the targeting agents described herein, the term “monovalent” is used to indicate that the targeting agent comprises a single binding domain; the term “bivalent” is used to indicate that the targeting agent comprises a total of two single binding domains; the term “trivalent” is used to indicate that the targeting agent comprises a total of three single binding domains; and so on for “multivalent” targeting agents. Alternatively, different targeting agents, directed against different binding sites, can be bound on one carrier, whereby each of the targeting agents can be mono-specific, monovalent, multispecific or multivalent.

In the composition described above, the carrier with the one or more targeting agents coupled, bound, linked or otherwise attached thereto or associated therewith may, for example, be maintained as a wettable powder, wettable granule, emulsifiable concentrate, suspension concentrate, microemulsion, capsule suspension, dry microcapsule, tablet or gel or be suspended, dispersed, emulsified or otherwise brought in a suitable liquid medium (such as water or another suitable aqueous, organic or oily medium) so as to provide a (concentrated) liquid composition hereof that has a stability that allows the composition hereof to be suitably stored or (where necessary after further dilution) applied to the intended site of action. Preferably, the composition hereof can be transported and/or stored prior to final use, optionally (and usually preferably) as a suitable liquid concentrate, dry powder, tablet, capsule suspension, slurry or “wet cake,” which can be suitably diluted, dispersed, suspended, emulsified or otherwise suitably reconstituted by the end user prior to final use. The composition hereof allows to be applied to the intended site of action using any suitable or desired manual or mechanical technique, such as, spraying, pouring, dripping, brushing, coating, drip-coating, applying as small droplets, a mist or an aerosol or any other suitable technique. Preferably, the intended site of action is an intact living insect, even more preferably an insect surface (as defined above). Upon such application to an insect or part of an insect, the carrier can bind to or at the binding site (or to one or more antigens present at or in the binding site or that form the binding site), preferably in a targeted manner (as described herein). In case more than one targeting agent is bound to a carrier, the targeting agents can be identical, or they can comprise different binding domains directed to the same or similar binding sites, or they may be directed to different binding sites. In one embodiment, one targeting agent may be directed to an antigen present in or at a binding site on the insect surface, whereas another targeting agent is directed towards an antigen present in or at a binding site on a place often frequented by the targeted insect. As a non-limiting example, one targeting agent may be targeting an antigen present at the surface of a plant pest insect species, while the other targeting agent is targeting an antigen present at the plant, which is the host plant of the pest insect species.

In the composition described above, the binding domain, comprised in the targeting agent, may be specific for one insect structure and/or one insect species, allowing selective binding and retaining of the carrier to one specific insect structure and/or one specific insect species, or it may be a broad spectrum binding domain, binding several insect structures and/or several insect species. In one non-limiting aspect hereof, the binding domain is binding specifically to a pest insect species, while it is not binding to benign insect species.

Still another aspect hereof is a formulation for controlling insect populations, comprising at least one targeting agent comprising at least one binding domain hereof. Formulations for controlling insect populations are known to the person skilled in the art and include, but are not limited to, liquid emulsifiable concentrates, wettable powders, solutions, suspension concentrates, emulsions, suspoemulsions, granules and water dispersible granules (Mulqueen, 2003). The binding domain may have an insecticidal activity by itself or it may exert its insecticidal activity by delivering and retaining an insecticide to an insect. Preferably, the formulation, according to the invention, further comprises an insecticide or a combination of insecticides. Even more preferably, the insecticide is bound on or comprised in a carrier. Preferably, the insecticide, more preferably the carrier comprising the insecticide, is coupled to the binding domain present in the formulation. Most preferably, the insecticide is covalently coupled to the targeting agent. In a preferred embodiment, the formulation is a pesticide formulation or an agrochemical formulation. A “pesticide formulation,” as used herein, means any composition comprising a compound or combination of compounds intended for preventing, destroying, repelling, attracting or mitigating any pest. An “agrochemical formulation,” as used herein, means a composition for agricultural use, comprising a biologically active agent, optionally with one or more additives favoring optimal dispersion, atomization, distribution, retention and/or activity of agrochemicals. As a non-limiting example such additives are diluents, solvents, adjuvants, surfactants, wetting agents, spreading agents, oils, stickers, penetrants, buffering agents, acidifiers, defoaming agents or drift control agents.

A further aspect hereof is a method for contacting an insect with a compound, preferably a biologically active agent, even more preferably an insecticide, the method comprising applying to or on sites frequented by insects a formulation comprising (a) at least one targeting agent comprising at least one binding domain according to the invention and (b) a compound. Preferably, the compound is bound on or comprised in a carrier, even more preferably, the compound, more preferably the carrier comprising the compound, is coupled, most preferably covalently coupled, to the targeting agent. The site, frequented by insects may be a natural habitat for insects, or a place regularly visited by insects. This site can be treated then with the formulation as described above: as a non-limiting example mosquito nets, impregnated with encapsulated insecticide, can be used as application method. Alternatively, the site is created by application of a visual lure or of an attractant for the insects. Visual lures are known to the person skilled in the art, and include but are not limited to, light sources, colored object and shapes or silhouettes that stand out of a contrasting background. As mentioned above, insect attractants include but are not limited to pheromones, kairomones and allomones. The attractant may be present in the formulation or it may be applied separately from the formulation, to ensure that the insects are attracted to the site where the formulation is applied.

Still another aspect hereof is a method to isolate the binding domain hereof, the method comprising selection of the binding domain using entire, intact insects. Preferably, the binding domain comprises an amino acid sequence comprised of 4 framework regions and 3 complementary determining regions. More preferably, the binding domain is derived from a camelid antibody, generated by immunization of a camelid with a whole insect extract. Most preferably, the binding domain is a binding domain according to the invention.

### EXAMPLES

### Example 1

### Generation and Selection of VHH

Immunization of Llamas with Insect Homogenates:

Colorado potato beetles (Leptinotarsa decemlineata) were dissected, exoskeletons and wings collected separately, and remainders discarded. Exoskeletons and wings were separately frozen in liquid nitrogen, ground with mortar and pestle, and fine powders collected. Colorado potato beetle larvae, Pea aphids (Acyrthosiphon pisum), and Tobacco Budworm larvae (Heliothis virescens), were frozen in liquid nitrogen, ground with mortar and pestle, and fine powders collected. Collected insect materials were resuspended in PBS and total protein concentrations of suspensions were determined with Bradford protein assay. Approximate total protein concentrations were 4.2, 0.3, 4.2, 2.7, and 2.3 mg/ml for Colorado potato beetle (CPB) exoskeletons, CPB wings, Pea aphids, CPB larvae, and Tobacco Budworm larvae suspensions, respectively. Suspensions were mixed on basis of equal total protein concentration and aliquots were prepared, stored at −80° C., and suspensions were used for immunization.

Two llamas, named Curley Sue and Jean Harlow, were immunized at weekly intervals with 6 intramuscular injections of mixed insect suspensions using Freund's Incomplete Adjuvant (FIA). Doses for immunizations were 125 μg total protein for days 0 and 6, and 62.5 μg total protein for days 13, 20, 27, and 34. At day 0 and at time of PBL collection at day 38 sera of llamas were collected.

Library Construction:

From each immunized llama a separate VHH library was made. RNA was isolated from peripheral blood lymphocytes, followed by cDNA synthesis using random hexamer primers and Superscript III according to the manufacturer's instructions (Invitrogen). A first PCR was performed to amplify VHH and VH DNA fragments using a forward primer mix [1:1 ratio of call001 (5′-gtcctggctgctcttctacaagg-3′) and call001b (5′-cctggctgctcttctacaaggtg-3′)] and reverse primer call002 (5′-ggtacgtgctgttgaactgttcc-3′). After separation of VH and VHH DNA fragments by agarose gel electrophoresis and purification of VHH DNA fragments from gel, a second PCR was performed on VHH DNA fragments to introduce appropriate restriction sites for cloning using forward primer A6E (5′-gatgtgcagctgcaggagtctggrggagg-3′(SEQ ID NO:_)) and reverse primer 38 (5′-ggactagtgcggccgctggagacggtgacctgggt-3′(SEQ ID NO:_)). The PCR fragments were digested using PstI and Eco91I restriction enzymes (Fermentas), and ligated upstream of the pIII gene in vector pMES3. The ligation products were ethanol precipitated according to standard protocols, resuspended in water, and electroporated into TG1 cells. Library sizes were at least 1E+08 independent clones for both libraries. Single colony PCR on randomly picked clones from the libraries was performed to assess insert percentages of the libraries. The libraries “Curley Sue” and “Jean Harlow” had ≧80% insert percentages of full-length clones. Libraries were numbered 44 and 45 for llamas “Curley Sue” and “Jean Harlow,” respectively. Phages from each of the libraries were produced using VCSM13 helper phage according to standard procedures.

Phage Selections Against Pea Aphid Extracts:

For selections against pea aphid extracts first optimum coating concentrations were determined using a serum titer ELISA. Total pea aphid homogenate as used for immunizations was diluted to 25 μg/ml total protein in PBS and 100 μl per well of 5-fold serial dilutions were used for coating of ELISA plates (Maxisorp, Nunc). Coatings were performed at 4° C. overnight or over weekend. Sera of llamas Curley Sue and Jean Harlow were used to determine optimum pea aphid extract concentration for coating and 25 and 0.25 μg/ml total protein were used for selections. Coatings were performed at 4° C. overnight or over weekend. Wells were washed 3 times with PBS/0.05%-Tween-20 and blocked with 5% skimmed milk in PBS (5% MPBS). Phage were diluted in 2.5% MPBS and approximately 1E+12 cfu were used for each well. After binding to the wells at room temperature for 2 hrs, unbound phages were removed by extensive washing with PBS/0.05%-Tween-20 and PBS. Bound phage were eluted at room temperature with 0.1 mg/ml trypsin (Sigma) in PBS for 30 min. Eluted phage were transferred to a polypropylene 96-well plate (Nunc) containing excess AEBSF trypsin inhibitor (Sigma). The titers of phage from target-coated wells were compared to titers of phage from blank wells to assess enrichments. Phages were amplified using fresh TG1 cells according to standard procedures. Enrichments in selection round 1 were approximately 8-fold for library 44 and approximately 50-fold for library 45. Enrichments in selection round 2 were ≧10 and ≧100-foldfor libraries 44 and 45, respectively.

Phage Selections Against Whole Pea Aphids:

Selections were performed against alive aphids at the start of selections. Approximately 10 adult pea aphids together with a few nymphs were collected per 1.5 ml tube and each library 44 and 45 was selected independently against whole aphids. Aphids were pre-incubated in 5% MPBS/0.05%-Tween-20 with head-over-head rotation at RT for 30 min. Aphids were collected using a 0.8 μm spin filter (Vivaclear) and transferred to 500 μl phage premixes containing approximately 4E+12 colony forming units (cfu) of phage. Unbound phage were washed away using 50 ml PBS/0.05%-Tween-20 for each wash with head-over-head rotation. Aphids were collected after each wash by allowing aphids to sink to the bottom of the tubes by gravitation. Supernatants with unbound phage were removed by decanting and pipetting and discarded. Bound phage were eluted by transferring aphids to 1.5 ml tubes with 500 μl per tube 0.1 mg/ml trypsin (Sigma) in PBS/0.05%-Tween-20 and incubation at room temperature with head-over-head rotation for 30 min Trypsin-eluted phage were transferred to tubes containing 25 μl of 5 mg/ml AEBSF trypsin inhibitor (Sigma) in PBS. A small portion of phage was used for serial dilutions in 2×TY media in a 96-well plate and log phase TG1 were added for infection. After infection at 37° C. for 20 min. 5 μl droplets were grown on LB agar plates containing glucose-2% and ampicillin-100 μg/ml. Phage output numbers were calculated from the colony spots and were 6E+06 and 4E+07 for libraries 44 and 45, respectively. Phages for second round selections were amplified using fresh TG1 cells according to standard procedures. Second round selections were performed similarly to selection rounds 1 but input phage numbers were approximately 1E+11 cfu and more washes were performed to remove unbound phage. Phage output numbers were 1.4E+06 4.0E+05 for libraries 44 and 45, respectively.

### Example 2

### Characterization of VHH

Single-Point Binding ELISA:

A single-point binding ELISA was used for clones from pea aphid extract selections to identify clones binding to pea aphid extracts. VHH-containing extracts for ELISA were prepared as follows. 96-well plates with 100 μl per well 2×TY, 2% glucose 100 μg/ml ampicillin were inoculated from the master plates and grown at 37° C. overnight. 25 μl per well of overnight culture was used to inoculate fresh 96-well deep-well plates containing 1 ml per well 2×TY; 0.1% glucose; 100 μg/ml ampicillin. After growing at 37° C. in a shaking incubator for 3 hrs, IPTG was added to 1 mM final concentration and recombinant VHH were produced during an additional incubation for 4 hrs. Cells were spun down by centrifugation at 3,000 g for 20 min. and stored at −20° C. overnight. Cell pellets were thawed, briefly vortexed, and 125 μl per well of room temperature PBS was added. Cells were resuspended on an ELISA shaker platform at room temperature for 15 min. Plates were centrifuged at 3,000 g for 20 min and 100 μl per well of VHH-containing extract was transferred to polypropylene 96-well plates (Nunc) and stored at −20° C. until further use.

Binding of clones from pea aphid extract selections was analyzed using ELISA plates coated with 100 μl per well of 25 μg/ml total protein pea aphid extract, prepared similarly as for selections. After coating at 4° C. overnight and continued coating at room temperature for 1 hr on the next day, plates were washed 3 times with PBS/0.05%-Tween-20 and blocked with 5% MPBS for 1.5 hrs. Plates were emptied and filled with 90 μl per well 1% MPBS. 10 μl of VHH-containing extract from each clone was added to (an) antigen-coated well(s) and a blank well. VHH were allowed to bind at room temperature for 1 hr and unbound VHH were removed by washing three times with PBS/0.05%-Tween-20. Bound VHH were detected with sequential incubations with monoclonal mouse anti-histidine antibodies (Abd Serotec) in 1% MPBS/0.05%-Tween-20 and rabbit anti-mouse IgG whole molecule antibodies conjugated with alkaline phosphatase (RaM/AP) (Sigma) in 1% MPBS/0.05%-Tween-20. Unbound antibodies were removed by washing three times with PBS/0.05%-Tween-20. The plates were washed an additional two times with PBS and 100 μl pNPP disodium hexahydrate substrate (Sigma) was added to each well. The absorbance at 405 nm was measured and the ratio of VHH bound to (a) target-coated well(s) and a non-target-coated well was calculated for each clone. From selections against pea aphid extract 24 of 92 (26%) clones had a ratio greater than 1.2 and these clones were analyzed further by sequencing.

Single Colony PCR and Sequencing:

Single colony PCR and sequencing was performed on ELISA positive clones from pea aphid extract selections as follows. Cultures from master plate wells with ELISA positive clones were diluted 10-fold in sterile water. 5 μl from these diluted clones were used as template for PCR using forward primer MP57 (5′-ttatgcttccggctcgtatg-3′(SEQ ID NO:_)) and reverse primer GIII (5′-ccacagacagccctcatag-3′(SEQ ID NO:_). PCR products were sequenced by Sanger-sequencing using primer MP57 (VIB Genetic Service Facility, University of Antwerp, Belgium). For clones from whole aphid selections single colony PCR was performed on clones after 1 or 2 rounds of selection without screening individual clones for binding. A limited number of clones was analyzed directly for correct insert size on 1.5% agarose gels and all clones with correct insert size were sequenced. Clones VHH 16H9, VHH 16G11, and few other clones were found by sequencing the ELISA positive clones from pea aphid extract selections. Clones VHH 14C1, VHH 14E7, VHH 14A10 were found by sequencing clones with correct insert size after whole aphid selections.

Antibody Production and Purification:

VHH were produced in E. coli suppressor strain TG1 or non-suppressor strain WK6 (Fritz et al., NucleicAcidsResearch, Volume 16 Number 14 1988) according to standard procedures. Briefly, colony streaks were made and overnight cultures from single colonies inoculated in 2×TY; 2% glucose; 100 μg/ml ampicillin. The overnight cultures were used to inoculate fresh cultures 1:100 in 2×TY; 0.1% glucose; 100 μg/ml ampicillin. After growing at 37° C. in a shaking incubator for 3 hrs, IPTG was added to a 1 mM final concentration and recombinant VHH were produced during an additional incubation for 4 hrs. Cells were spun down and resuspended in 1/50^{th} of the original culture volume of periplasmic extraction buffer (50 mM phosphate pH7; 1M NaCl; 1 mM EDTA) and incubated with head-over-head rotation at 4° C. overnight. Spheroplasts were spun down by centrifugation at 3,000 g and 4° C. for 20 min Supernatants were transferred to fresh tubes and centrifuged again at 3,000 g and 4° C. for 20 min. Hexahistidine-tagged VHH were purified from the periplasmic extract using 1/15^{th} of the extract volume of TALON metal affinity resin (Clontech), according to the manufacturer's instructions. Purified VHH were concentrated and dialyzed to PBS using Vivaspin 5 kDa molecular weight cut-off (MWCO) devices (Sartorius Stedim), according to the manufacturer's instructions.

### Example 3

Binding of VHH to Intact Pea Aphids

Whole Insect ELISA:

To analyze binding of selected VHH to insects a whole aphid ELISA was developed (see FIG. 1). Aphids were collected and washed by head-over-head rotation in PBS. Aphids were dispensed in wells of a 96-well deep-well filtration plate (Millipore) and incubated in 1 ml PBS on an ELISA shaker for 20 min. Two wells were used for each VHH to be screened. PBS was drained from the wells using a vacuum manifold (Millipore). Purified VHH were diluted to 5 μg/ml in PBS/1%-BSA and 250 μl of solutions containing anti-insect VHH, control VHH, or PBS/1%-BSA alone were added to each well and incubated on an ELISA shaker at room temperature for 1 hr. Six to eight blanks were included in each experiment to account for specimen-to-specimen variation. Solutions of VHH or only PBS/1%-BSA were drained from the wells and each well was washed five times with PBS. For each wash 1 ml per well PBS was added, incubated for 2 min. on an ELISA shaker, and drained using the vacuum manifold. Bound VHH were detected with sequential incubations with monoclonal mouse anti-histidine antibodies (Abd Serotec) in PBS/1%-BSA and rabbit anti-mouse IgG whole molecule antibodies conjugated with alkaline phosphatase (RaM/AP) (Sigma) in PBS/1%-BSA. Unbound antibodies were removed by washing five times with PBS. pNPP disodium hexahydrate substrate (Sigma) was added to each well and incubated for 30 min. Colored substrates were collected using the filtration plate setup using a deep-well collector plate and transferred to an optical plate. The absorbance at 405 nm was measured and compared to the average absorbance of the blank wells. The whole aphid ELISA was performed at least two times on different days for each clone. Measured absorbance for clones VHH 14C1, VHH 14E7, VHH 14A10, VHH 16H9, and VHH 16G11 was consistently above blank for each aphid measured and these clones showed ratios over blank between 1.4 and 3.6 (see Table 1).

TABLE 1
Measured absorbance for clones VHH 14C1, VHH 14E7, VHH
14A10, VHH 16H9, and VHH 16G11 was consistently above
blank for each aphid measured in a whole aphid ELISA.

| | Screen#1 | | | Screen#2 | | |
|---|---|---|---|---|---|---|
| | Aphid 1 | Aphid 2 | Average | Aphid 1 | Aphid 2 | Average |
| VHH14C1 | 0.484 | 0.476 | 0.480 | 0.775 | 0.842 | 0.809 |
| VHH14E7 | 0.664 | 0.584 | 0.624 | 1.131 | 0.828 | 0.980 |
| VHH14A10 | 1.186 | 1.289 | 1.238 | 2.054 | 1.765 | 1.910 |
| VHH16G11 | 0.601 | 0.809 | 0.705 | No data | No data | No data |
| VHH16H9 | 0.667 | 0.583 | 0.625 | 0.785 | 0.929 | 0.857 |
| Blank | | | 0.345 | | | 0.586 |

Microscopic Analysis:

To visualize binding of selected VHH to insects, whole aphids were labeled with purified VHH. Aphids were collected and washed by head-over-head rotation in PBS. One aphid per 0.5 ml tube was used and three tubes for each VHH to be analyzed. PBS was removed from the tubes using a pipette. Purified VHH were diluted to 5 lag/ml in PBS/1%-BSA and 250 μl of solutions containing anti-insect VHH, control VHH, or PBS/1%-BSA alone were added to each tube and incubated with head-over-head rotation at room temperature for 1 hr. Solutions of VHH or only PBS/1%-BSA were removed from the tubes and each tube was washed five times with PBS. For each wash 0.5 ml PBS per tube was added, incubated for 2 min with head-over-head rotation, and supernatant removed by pipetting. Bound VHH were detected with sequential incubations with monoclonal mouse anti-histidine antibodies (Abd Serotec) in PBS/1%-BSA and rabbit anti-mouse IgG conjugated with Alexa594 (RaM/Alexa594) (Invitrogen) in PBS/1%-BSA. Unbound antibodies were removed by washing five times with PBS. Aphids were placed in 18-well μ-slides and analyzed by microscopy. Clear binding of anti-insect VHH to the surface of aphids was demonstrated (FIG. 2).

### Example 4

### Targeting to Insects

With the objective to generate VHH-functionalized polyurea microcapsules, VHH were coupled to microcapsules with a core of 1.5% Uvitex OB (source) in benzyl benzoate and a shell with incorporated lysine to surface-expose carboxyl groups. A core of 1.5% Uvitex OB in benzyl benzoate was used for fluorescent visualization of microcapsules. After production of microcapsules, microcapsules were washed with water and stored as capsule suspensions in water. Before coupling of VHH, microcapsules were washed with MES/NaCl buffer (0.1 M MES/0.5 M NaCl pH 6) using a 96-well deep-well filtration plate (Millipore) and vacuum manifold (Millipore). Insect-binding VHH in PBS were then added to a final concentration of 16 μM and incubated with the microcapsules for 15-30 min 1-Ethyl-3-[3-dimethylaminopropyl]carbodiimide Hydrochloride (EDC) (Pierce) was dissolved in MES/NaCl buffer and promptly added to a final concentration of 50 mM. VHH were coupled by incubation with continuous mixing at room temperature for 2 hrs. The coupling reaction was stopped by adding Tris-buffer pH 7.5 to a final concentration of 50 mM and incubation at room temperature for 30 min. Non-bound VHH were collected using the filtration plate setup using a deep-well collector plate to calculate coupling efficiency. Microcapsules were washed with PBS and resuspended in PBS and stored at 4° C. until use. To demonstrate that coupling of insect-binding VHH functionalizes microcapsules a binding experiment with microcapsules to pea aphids was performed. For this purpose aphids were incubated with VHH-coupled microcapsules, blank microcapsules, or without microcapsules. Aphids were incubated with microcoapsules to allow microcoapsule binding to aphids. Unbound microcapsules were washed away from the aphids by careful pipetting to resuspend microcapsules and supernatants were discarded. Aphids were transferred to microscope object glasses and analyzed microscopically. Clear binding of microcapsules to aphid bodies and legs were observed (see FIG. 3).

In conclusion, VHH binding to insects can be isolated using appropriate methodologies and these VHH are resultingly binding their epitopes on native targets and can be used for targeting of compounds to their site of action.

### REFERENCES

Altschul, S. F., Madden, T. L., Schäffer, A. A., Zhang, J., Zhang, Z., Miller, W. and Lipman, D. J. (1997). Gapped BLAST and PSI-BLAST: a new generation of protein database search programs, Nucleic Acids Res. 25:3389-3402.
Dimitrov, D. S. (2009) Engineered CH2 domains (nanoantibodies). mAbs 1, 26-28.
Doctor, J., Fristrom, D., and Frsitrom, J. W. (1985). The pupal cuticle of Drosophila: biphasic synthesis of pupal cuticle proteins in vivo and in vitro in response to 20-hydroxyecdysone. J. Cell. Biol. 101, 189-200.
Hamel, F., Boivin, R., Tremblay, C. and Bellemare, G. (1997). Structural and evolutionary relationships among chitinases of flowering plants. J. Mol. Evol. 44, 614-624.
Iseli, B., Boller, T. and Neuhaus, J. M. (1993). The N-terminal cysteine-rich domain of Tobacco Class I chitinase is essential for chitin binding but not for catalytic or antifungal activity. Plant Physiol. 103, 221-226.
Kenawy, E. R. (1998). Recent advantages in controlled release of agrochemicals. Polymer reviews 38, 365-390.
Knight, A. L. (2010). Targeting Cydia pomonella (L.) (Lepidoptera: Tortricidae) adults with low-volume application of insecticides alone and in combination with sex pheromone. Pest Manag. Science 66: 709-717.
Mulqueen, P. (2003). Recent advantages in agrochemical formulation. Adv. Coll. Interface Sci. 106, 83-107.
Nutall, S. D., Krishnan, U. V., Doughty, L., Pearson, K., Ryan, M. T., Hoogenraad, N. J., Hattakari, M., Carmichael, J. A., Irving, R. A. and Huson, P. J. (2003). Isolation and characterization of an IgNAR variable domain specific for the human mitochondrial translocase receptor Tom70. Eur. J. Biochem. 270, 3543-3554.
Perrin, B. (2000). Improving insecticides through encapsulation. Pesticide Outlook, 11, 68-71.
Rebers, J. E. and Willis, J. H. (2001). A conserved domain in arthropod cuticular proteins binds chitin. Insect Biochem. Molecul. Biol. 31, 1083-1093.
Sales, M. P., Pimenta, P. P., Paes, N. S., Grossi-de-Sa, M. F. and Xavier-Filho, J. (2001). Vicilins (7S storage globulins) of cowpea (Vigna unguiculata) seeds bind to chitinous structures of the midgut of Callosobruchus maculates (Coleoptera: Bruchidae) larvae. Braz. J. Med. Biol. Res. 34, 27-34.
Tramontano, A., Bianchi, E., Venturini, S., Martin, F., Pessi, A. and Sollazzo, M. (1994) The making of the minibody: an engineered beta-protein for the display of confromationally constrained peptides. J. Mol. Recognition. 7, 9-24.
Wesolowski, J., Alzogaray, V., Reyelt, J., Unger, M., Juarez, K., Urrutia, M., Cauerhiff, A., Danquah, W., Rissiek, B., Scheuplin, F., Schwarz, N., Adriouch, S., Boyer, O., Seman, M., Licea, A., Serreze, D. V., Goldbaum, F. A., Haag, F. and Koch-Nolte, F. (2009). Single domain antibodies: promising experimental and therapeutic tools in infection and immunity. Med. Microbiol. Immunol. 198, 157-174.

1. A binding domain able to bind a binding site on an insect.

2. The binding domain of claim 1, wherein the binding site is situated on the surface of the insect.

3. The binding domain according to claim 2, wherein said binding site is situated on the insect exoskeleton.

4. The binding domain of claim 2, wherein the binding site is comprised in an insect structure selected from the group consisting of head, thorax, abdomen, trachea, spiracles, antennae, legs, claws, wings, wingshells, mouthparts, eyes of the insect or any combination thereof.

5. The binding domain of claim 1, wherein the binding domain comprises a peptide comprising four (4) framework regions and three (3) complementary determining regions, or any suitable fragment thereof.

6. The binding domain of claim 5, wherein the binding domain is from a camelid antibody.

7. The binding domain according to claim 6, wherein said binding domain is comprised in a VHH sequence.

8. The binding domain of claim 7, wherein the VHH comprises one of SEQ ID NO:1 through SEQ ID NO:5.

9. The binding domain of claim 1 able to retain a carrier on an insect.

10. The binding domain of claim 9, wherein the binding domain comprises a peptide comprising four (4) framework regions and three (3) complementary determining regions.

11. The binding domain of claim 1 able to retain a compound on an insect.

12. The binding domain of claim 11, wherein the binding domain comprises a peptide comprising 4 framework regions and 3 complementary determining regions.

13. The binding domain of claim 1, wherein the binding domain is coupled to an insecticide.

14. The binding domain according to claim 13, wherein said insecticide is bound on or comprised in a carrier.

15. A method of delivering a compound to and/or retaining a compound on an insect, the method comprising:
utilizing a targeting agent comprising at least one binding domain of claim 1 so as to deliver and/or retain the compound to the insect.

16. The method according to claim 15, wherein the compound is bound on or comprised in a carrier.

17. A method of modifying a target specificity of a compound, the method comprising:
utilizing a targeting agent comprising at least one binding domain of claim 1 so as to modify the target specificity of the compound.

18. A composition, comprising a targeting agent comprising at least one binding domain of claim 1 and a carrier.

19. A formulation for controlling insect populations, wherein the formulation comprises at least one targeting agent comprising at least one binding domain of claim 1.

20. A formulation for controlling insect populations, wherein the formulation comprises:
at least one targeting agent comprising at least one binding domain comprising a peptide comprising 4 framework regions and 3 complementary determining regions, or any suitable fragment thereof.

21. The formulation of claim 19, further comprising an insecticide.

22. The formulation according to claim 21, wherein said insecticide is bound on or comprised in a carrier.

23. A method for contacting an insect with a compound the method comprising:
applying to or on more sites frequented by insects, a formulation comprising:
at least one targeting agent comprising at least one binding domain of claim 1, and
the compound.

24. A method for isolating a binding domain, the method comprising:
selection of the binding domain on at least one intact insect.

25. The method according to claim 24, wherein the binding domain comprises a peptide comprising four (4) framework regions and three (3) complementary determining regions, or any suitable fragment thereof.


SEQUENCE LISTING (12 sequences)

SEQ ID NO: 1 - protein, 120 aa, Lama glama
  MISC_FEATURE: 14A10
QVQLQESGGGLVQAGGSLKLSCAAAIRTFSILNMGWFRQAPGKEREFVATINRSGATTYYADSVKGRFAISRDNAKNTMYLQMNSLKPEDTAVYYCTADLWRIRGAMDYWGKGTQVTVSS

SEQ ID NO: 2 - protein, 124 aa, Lama glama
  MISC_FEATURE: 14C1
QVQLQESGGGLAQPGGSLTLSCKVTGSTLDYYAIGWYRQAPGKEREGVSCISSRDATNYEDSVKGRFTISRDTVKNTVYLQMRNLKPEDTAVYYCAAASRYVSISGIYCPADRWGQGTQVTVSS

SEQ ID NO: 3 - protein, 114 aa, Lama glama
  MISC_FEATURE: 14E7
QVQLQESGGGLVQPGGSLKLSCAASGFAFSFLRMGWYRQAPGKQRELVAYITSGGSTNYIDSVKGRFTISRDNAKNTMYLQMNSLKPEDTARYYCNEYPQSNSWGQGTQVTVSS

SEQ ID NO: 4 - protein, 111 aa, Lama glama
  MISC_FEATURE: 16G11
QVQLQESGGGLVQPGGSLRLSCAASGSVINFNFMRWYRQVPGNQREWVAIINSGGSTYYRDSVKGRFTISRDNAKNTVYLQMNSLKPEDTAVYYCNAENTWGQGTQVTVSS

SEQ ID NO: 5 - protein, 111 aa, Lama glama
  MISC_FEATURE: 16H9
QVQLQESGGGLVQPGGSLRLSCAASGSIISFNFMRWYRQVPGNQREWVAIINSSGSTYYRDSVKGRFTISRDNAKNTVYLQMNSLKPEDTAVYYCNAENIWGQGTQVTVSS

SEQ ID NO: 6 - DNA, 23 bp, Artificial Sequence
  Primer
gtcctggctgctcttctacaagg

SEQ ID NO: 7 - DNA, 23 bp, Artificial Sequence
  Primer
cctggctgctcttctacaaggtg

SEQ ID NO: 8 - DNA, 23 bp, Artificial Sequence
  Primer
ggtacgtgctgttgaactgttcc

SEQ ID NO: 9 - DNA, 29 bp, Artificial Sequence
  Primer
gatgtgcagctgcaggagtctggrggagg

SEQ ID NO: 10 - DNA, 35 bp, Artificial Sequence
  Primer
ggactagtgcggccgctggagacggtgacctgggt

SEQ ID NO: 11 - DNA, 20 bp, Artificial Sequence
  Primer
ttatgcttccggctcgtatg

SEQ ID NO: 12 - DNA, 19 bp, Artificial Sequence
  Primer
ccacagacagccctcatag